(19) United States
(12) Patent Application Publication
(10) Pub. No.: US 2021/0369895 A1
(43) Pub. Date: Dec. 2, 2021

(54) UV SANITIZING CABINET

(71) Applicant: NSX Technologies, Inc. DBA Anywhere Cart, Temecula, CA (US)

(72) Inventor: William Todd McKelvie, Temecula, CA (US)

(21) Appl. No.: 17/336,021

(22) Filed: Jun. 1, 2021

Related U.S. Application Data
(60) Provisional application No. 63/032,957, filed on Jun. 1, 2020.

(51) Int. Cl. A61L 2/10

(52) U.S. Cl. CPC A61L 2/10; A61L 2202/122; A61L 2202/11

(57) ABSTRACT

A UV sanitizing cabinet includes outer walls and a control panel located thereon. An inner enclosure is defined by inner walls and a pivotally mounted door that may be selectively moved between an open orientation and a closed orientation. An upper rack and a lower rack are selectively positioned within the enclosure. At least one UV lamp is positioned below the lower rack within the enclosure and at least one UV lamp is positioned above the upper rack within the enclosure. A middle shelf is selectively positioned within the enclosure. Tracks are secured along the inner walls for supporting the upper and lower racks, as well as the middle shelf, in a manner allowing for selective removal and insertion thereof such that the cabinet can support various electronic devices with minimal contact, including, those with a hinge in an open 180 degree orientation.

### BACKGROUND OF THE INVENTION

### 1. Field of the Invention

The present invention relates to UV (ultraviolet) sanitizing cabinets.

### 2. Description of the Related Art

The use of laptops and tablets in classrooms has expanded tremendously in recent times. As the laptops and tablets are commonly used by multiple students during the course of a day, problems associated with the transfer of diseases/germs from one student to another student are common. Electronic devices such as laptops and CHROMEBOOKS® include a hinge so that they may be open and closed. However, when closed, the keyboard and monitor cannot be sanitized.

The present sanitizing cabinet offers a mechanism for storing open laptops and tablets in a classroom environment while also providing for the sterilization of all surfaces thereof. When all surfaces of the laptops and tablets have been sterilized, they then can be transferred amongst the many students making use of them during the course of the day without the passing of germs.

### SUMMARY OF THE INVENTION

It is, therefore, an object of the present invention to provide a UV sanitizing cabinet including outer walls and a control panel located thereon. An inner enclosure is defined by inner walls and a pivotally mounted door that may be selectively moved between an open orientation and a closed orientation. An upper rack and a lower rack are selectively positioned within the enclosure. At least one UV lamp is secured below the lower rack within the enclosure and at least one UV lamp is secured above the upper rack within the enclosure. A middle shelf is selectively positioned within the enclosure. Tracks are secured along the inner walls for supporting the upper and lower racks, as well as the middle shelf, in a manner allowing for selective removal and insertion thereof such that the cabinet can support electronic devices with minimal contact, for example, a laptop fully opened along its hinge in an open 180 degree orientation.

It is also an object of the present invention to provide a UV sanitizing cabinet wherein the inner walls include a left side inner wall, a right side inner wall, an upper inner wall, a lower inner wall, and a back inner wall all made from a highly reflective metal.

It is also an object of the present invention to provide a UV sanitizing cabinet wherein the at least one UV lamp positioned below the lower rack includes a plurality of staggered lower UV lamps secured along the lower inner wall.

It is also an object of the present invention to provide a UV sanitizing cabinet wherein the at least one UV lamp positioned above the upper rack includes a plurality of staggered upper UV lamps secured along the upper inner wall.

It is also an object of the present invention to provide a UV sanitizing cabinet wherein the at least one UV lamp secured above the upper rack includes a plurality of upper UV lamps secured along the upper inner wall.

It is also an object of the present invention to provide a UV sanitizing cabinet wherein the upper rack, the lower rack, and the middle shelf are made from cylindrical wire.

It is also an object of the present invention to provide a UV sanitizing cabinet wherein the tracks include upper, lower, and middle tracks attached along the left and right inner walls upon which the racks and shelf travel.

It is also an object of the present invention to provide a UV sanitizing cabinet wherein the upper rack and the lower rack are identical and interchanging.

It is also an object of the present invention to provide a UV sanitizing cabinet wherein the upper rack and/or the lower rack include a rectangular wire frame with a plurality of upstanding supports attached along a top of the rectangular wire frame.

It is also an object of the present invention to provide a UV sanitizing cabinet wherein the middle shelf includes a rectangular wire frame.

It is also an object of the present invention to provide a UV sanitizing cabinet further including a locking access door on an outer side wall so that a door lock can easily be accessed if needed.

It is also an object of the present invention to provide a UV sanitizing cabinet wherein the control panel includes a change UV lamp feature which displays a change light message.

It is also an object of the present invention to provide a UV sanitizing cabinet wherein the plurality of upstanding supports include a single top bar at the peak and upstanding side bars which are bent inwardly prior to respective ends of the single top bar.

It is also an object of the present invention to provide a UV sanitizing cabinet wherein the plurality of upstanding supports further include riser bars that form a “V” and respectively intersect the top bar at the point that the upstanding side bars intersects the top bar.

Other objects and advantages of the present invention will become apparent from the following detailed description when viewed in conjunction with the accompanying drawings, which set forth certain embodiments of the invention.

### BRIEF DESCRIPTION OF THE DRAWINGS

FIG. 1 is a front perspective view of a UV sanitizing cabinet with the door open and the shelves in a configuration for receiving tablets.

FIG. 2 is a front elevation view of the UV sanitizing cabinet shown in FIG. 1.

FIG. 3 is a front perspective view of the UV sanitizing cabinet with the door open and the shelves in a configuration to receive laptops.

FIG. 4 is a front perspective view of the UV sanitizing cabinet from below as shown in FIG. 3.

FIG. 5 is a front perspective view of the UV sanitizing cabinet filled with laptops and with the door open.

FIG. 6 is a perspective view of a plurality of closed laptops.

FIG. 7 is a front perspective view of the UV sanitizing cabinet filled with tablets and with the door open.

FIG. 8 is a front perspective view of the UV sanitizing cabinet half filled with tablets.

FIG. 9 is a front perspective view of an alternate embodiment of the UV sanitizing cabinet.

FIG. 10 is a front elevation view of the UV sanitizing cabinet shown in FIG. 9.

FIG. 11 is a perspective view of an alternate rack embodiment (which, although referenced as the lower rack, applies equally to the upper rack).

### DESCRIPTION OF THE PREFERRED EMBODIMENTS

The detailed embodiments of the present invention are disclosed herein. It should be understood, however, that the disclosed embodiments are merely exemplary of the invention, which may be embodied in various forms. Therefore, the details disclosed herein are not to be interpreted as limiting, but merely as a basis for teaching one skilled in the art how to make and/or use the invention.

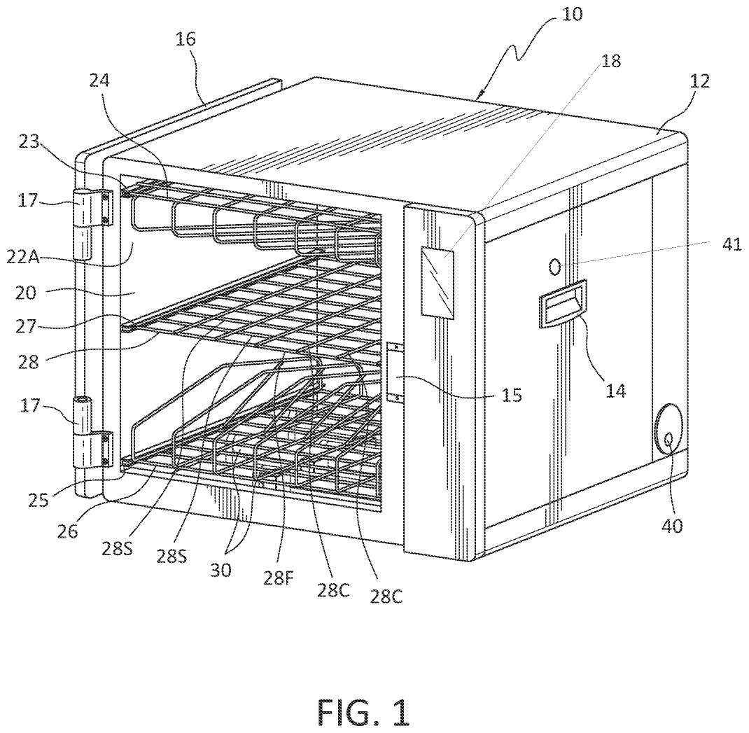

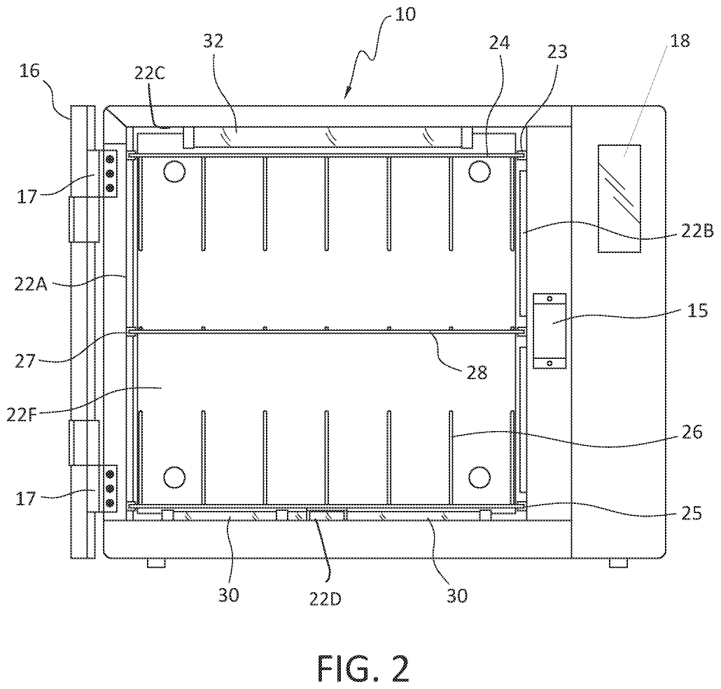

Referring to FIGS. 1-5 and 7-11, various views of the Ultraviolet (UV) sanitizing cabinet of the present invention are disclosed.

In accordance with a disclosed embodiment as shown with reference to FIGS. 1-8, the cabinet 10 is constructed from metal. The cabinet 10 has outer walls 12 with handles 14 on opposite sides thereof and a digital control panel 18 located on the front thereof. The cabinet outer wall 12 further includes an opening 40 for a power supply cord (not shown).

The cabinet 10 has an inner enclosure 20 with inner walls 22A-E made from highly reflective aluminum or stainless steel sheeting. In particular, the inner enclosure is defined by a left side inner wall 22A, a right side inner wall 22B, an upper inner wall 22C, a lower inner wall 22D, and a back inner wall 22E. The inner enclosure 20 is further defined by a pivotally mounted cabinet door 16 that may be selectively moved between an open orientation and a closed orientation.

The inner enclosure 20 includes a plurality of lower UV lamps 30 secured along the lower inner wall 22D. In accordance with a disclosed embodiment, four lower UV lamps 30 are provided and are positioned in a staggered relationship (that is, the UV lamps extend left to right, wherein two UV lamps are offset to the left of center and two UV lamps are offset to the right of center) along the lower inner wall 22D. It is appreciated the UV lamps may include one or two replaceable bulbs.

The inner enclosure 20 also includes a plurality of upper UV lamps 32 which may include one or two replaceable bulbs. The upper UV lamps 32 are located within or adjacent to the upper inner wall 22C. In accordance with a disclosed embodiment, four upper UV lamps 32 are provided and are positioned in an aligned relationship (that is, the UV lamps extend left to right, wherein all four UV lamps are centrally aligned) along the upper inner wall 22C.

Upper and lower racks 24, 26, respectively, are provided for selective positioning within the inner enclosure 20. The racks 24, 26 are made from medical grade stainless steel wire. The inner enclosure 20 further includes a selectively removable middle shelf 28 located between the upper rack 24 and the lower rack 26. In accordance with a disclosed embodiment, the middle shelf 28 is also be made from medical grade stainless steel wire.

As mentioned above, the upper rack 24, the lower rack 26, and the middle shelf 28 are all selectively removable and insertable. Selective removal and insertion of the upper rack 24, the lower rack 26, and the middle shelf 28 is achieved by the provision of upper, lower, and middle tracks 23, 25, 27 respectively attached along the left and right inner walls 22A, 22B which allow for the upper rack 24, the lower rack 26, and the middle shelf 28 to slide in and out of the enclosure 20 along the slots form in the tracks 23, 25, 27. In particular, the upper rack 24 travels in the slots defined by the upper tracks 23, the lower rack 26 travels in slots defined by the lower tracks 25, and the middle shelf 28 travels in the slots defined by the middle tracks 27. The upper rack 24 and the lower rack 26 are selectively removable so that the UV bulbs in UV lamps 30, 32 can be replaced (as it is appreciated they will have a limited life). As will be appreciated based upon the following disclosure, the middle shelf 28 is selectively removable to allow for use of the cabinet 10 with articles of different sizes.

The upper rack 24 and the lower rack 26 are identical and thus can be interchanged. Accordingly, only the lower rack 26 will be described in detail. The lower rack 26 includes a rectangular wire frame 26F with a series of spaced cross bars 26C. The cross bars 26C are sufficiently spaced to allow the UV light to pass through the lower rack 26.

A plurality of upstanding supports 29 are attached along the top of the rectangular wire frame 26F and spaced cross bars 26C such that the upstanding supports 29 lie in plane that is perpendicular to the plane in which the rectangular wire frame 26F lies.

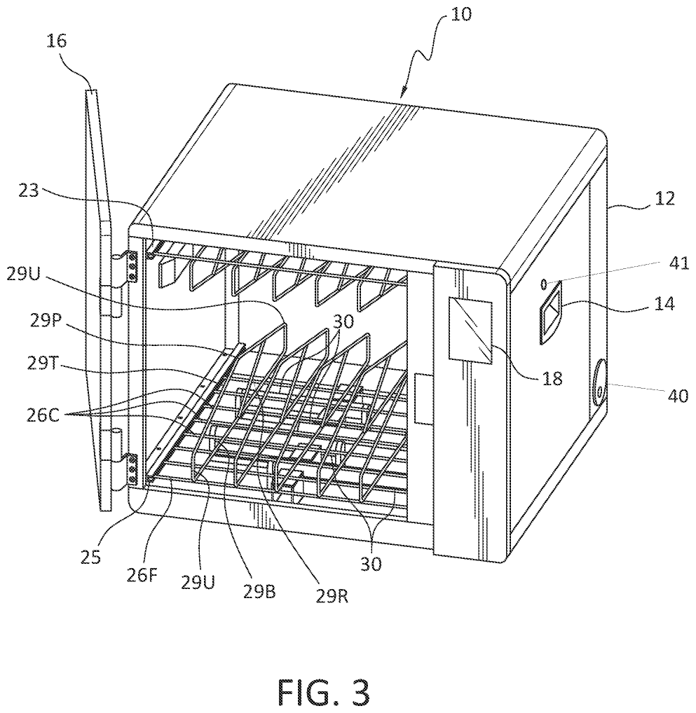

Each of the upstanding support 29 is specifically shaped to support devices of various dimensions in need of sanitation. In accordance with a disclosed embodiment, each of the upper and lower racks 24, 26 includes a series of seven spaced upstanding supports 29 that support articles held by the cabinet 10 (although the number of supports may be varied as shown with reference to FIGS. 3, 4, 9, 10, and 11).

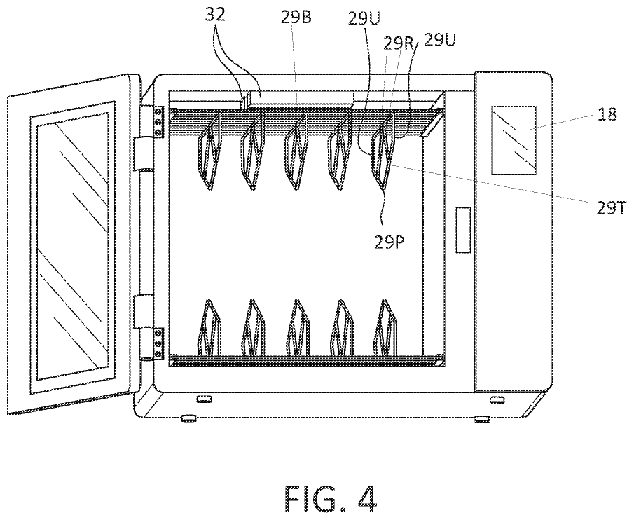

Referring to the embodiments of the upstanding supports as disclosed with reference to FIGS. 1-10, the upstanding supports 29 of the upper and lower racks 24, 26 are substantially identical and one of the upstanding supports is described. The upstanding support 29 is generally gable shaped (much like a home with a gabled roof). That is, the upstanding support 29 includes a bottom bar 29B which is lies directly upon and is secured to the wire frame 26F. The bottom bar 29B extends into an upstanding side bars 29U at the first and second ends thereof. The upstanding side bars 29U extend upwardly from the wire frame 26F, preferably at an angle perpendicular to the plane in which the wire frame 26F lies. Each of the upstanding side bars 29U continues into a top bar 29T. The top bars 29T are oriented at an obtuse angle relative to the upstanding side bars 29U and extend from the upstanding side bars 29U such that the top bars 29T ultimately lie in the same plane as the other elements of the upstanding support 29. The top bars 29T taper upwardly until they meet at a peak 29P of the upstanding support 29. Each upstanding support 29 further includes at least one riser bar 29R (for example, FIGS. 1, 3, 5, 7, and 8 show embodiments with a single riser bar, whereas FIGS. 4, 9, and 11 show embodiments with two riser bars) which extends from the bottom bar 29B at an angle to one side of the top bar 29T. As briefly mentioned above, this shape permits tablets, laptops, CHROMEBOOKS®, smartphones, and various other electronic devices in need of sanitation to be supported within the inner enclosure 20.

Considering the identical nature of the upper rack 24 and the lower rack 26, the upstanding supports 29 thereof laterally align when the upper and lower racks 24, 26 are installed within the inner enclosure 20. In particular, with the upper and lower racks 24, 26 installed within the inner enclosure 20, the upstanding supports 29 of the upper rack 24 extend downwardly within the inner enclosure 20 and the upstanding supports 29 of the lower rack 26 extend upwardly within the inner enclosure 20 such that the respective peaks 29P of the aligned upstanding supports 20 point toward each other. This arrangement creates elongated spaces in which articles may be positioned. For example, and considering the arrangement where the middle shelf 28 is removed, the elongated spaces are ideally arranged to accept and support fully opened laptops.

Enhanced support of the laptops, tablets, as well as other articles that may be placed within the inner enclosure 20, is achieved as a result of the cylindrical, thin gauged, wire from which the upstanding supports 29 are constructed and the specific construction employed in the fabrication of the upstanding supports 29. The cylindrical, thin gauged, wire minimizes the surface area of the upstanding supports 29 and thereby minimizes blockage of UV rays as they are transmitted within the inner enclosure 20 and interact with surfaces of the articles. Further, the provisions of gable shape also minimize blockage of UV rays as they interact with surfaces of the article by minimizing the contact between the articles and the upstanding supports 29. For example, when a tablet is inserted and leaned against an upstanding support 29, only the portion of the tablet contacting the peak 29P will actually be in contact with the upstanding support 29. Also, due to the peak 29P being made from a cylindrical metal, the contact with the tablet is further reduced as only a portion of the circle will contact the tablet.

The middle shelf 28 includes a rectangular wire frame 28F having a series of cross bars 28S which extend within the frame 28F from side to side and a second series of spaced cross bars 28C which extend from front to rear.

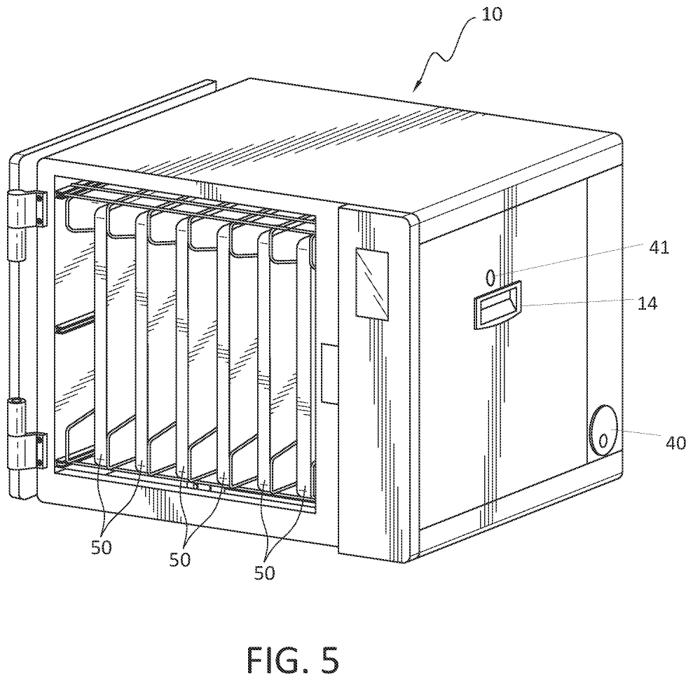

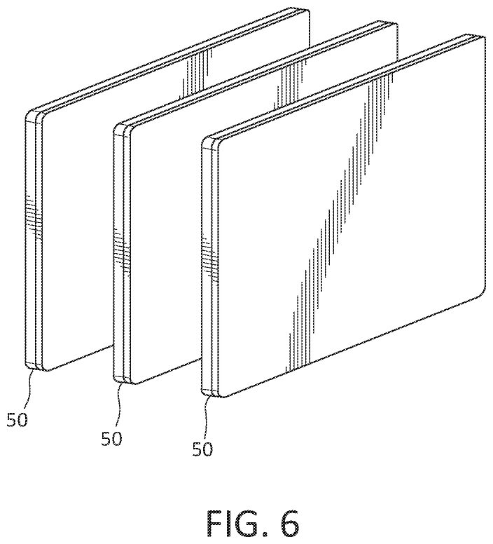

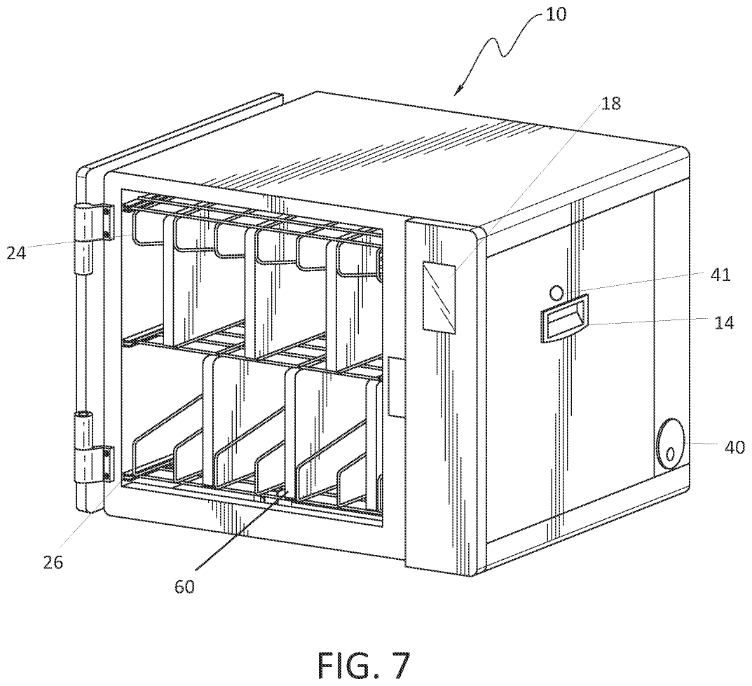

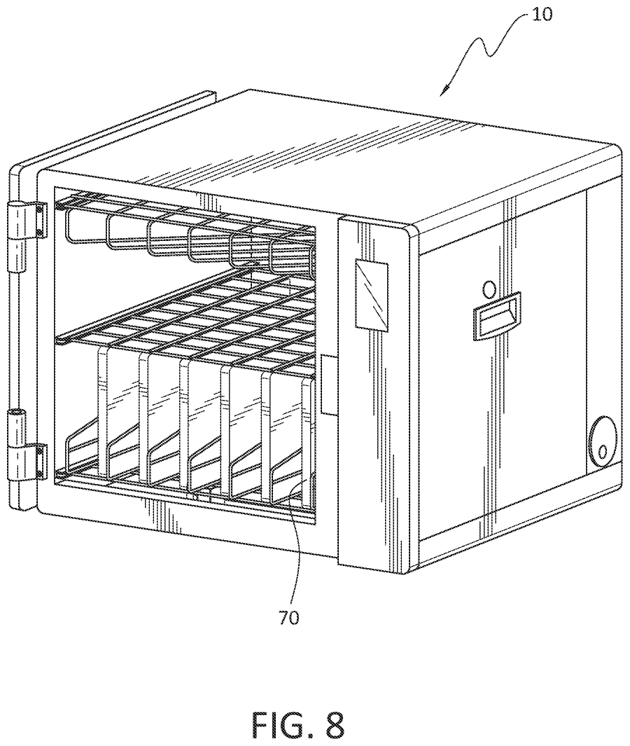

As shown in FIG. 5, with the middle shelf 28 removed the cabinet 10 can hold six laptops 50 in the 180-degree position shown in FIG. 6, that is when the laptop is open and flat with the keyboard and monitor at approximately 180-degrees. This 180-degree position permits all surfaces of the laptops 50 to be sanitized. As shown in FIG. 7, with the middle shelf inserted the cabinet 10 can hold twelve tablets 60 to be sanitized. As shown in FIGS. 7 and 8, the cabinet 10 can support either tablets 60 of varying dimensions and/or smartphones 70 of varying dimensions.

Considering the desire to store and sanitize fully opened laptops, it is important that the inner enclosure 20 be of a size sufficient to accommodate a fully opened laptop. As such, a disclosed embodiment includes an inner enclosure 20 have the following dimensions: approximately 16.25 inches—Width; approximately 20.375 inches—Depth; and approximately 15.25 inches—Height.

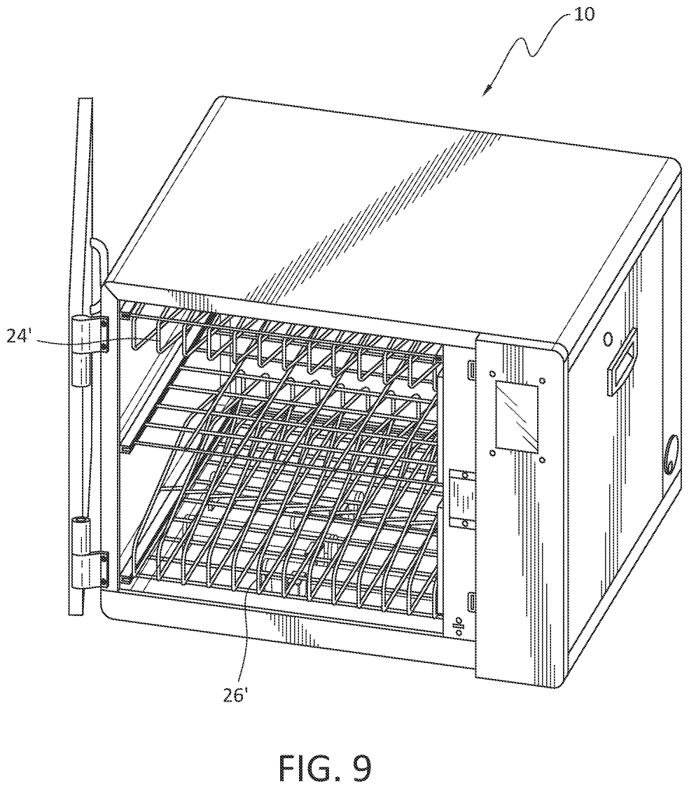

As shown in an alternate embodiment with reference to FIGS. 9 to 11, the cabinet 10 includes upper rack 24′ and lower rack 26′ which include thirteen upstanding supports 29′. When using upper rack 24′ in combination with lower rack 26′ the number of laptops that can held in the 180-degree position is increased to twelve. These racks 24 and 26 are interchangeable with racks 24′ and 26′ and 26″.

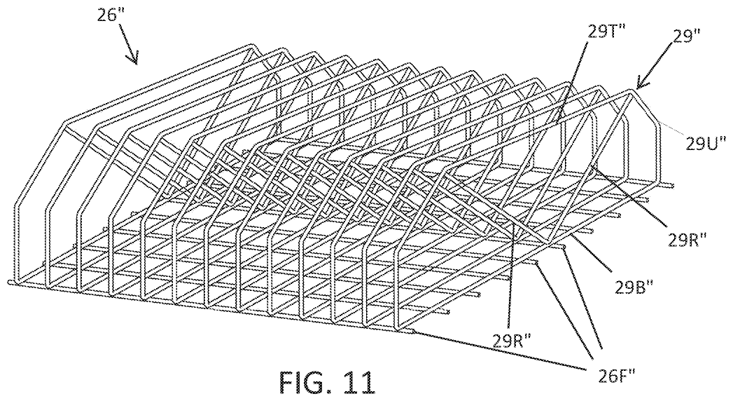

Further, and with regard to FIG. 11, the configuration of upstanding supports 29″ is modified. In accordance with the disclosed embodiment, the top bars do not meet at a peak, but rather the top bars of the prior embodiment are replaced with a single top bar 29T″ at the peak and the upstanding side bars 29U″ are bent inwardly prior to the respective ends of the single top bar 29T″. This embodiment also includes riser bars 29R″ that form a “V” that respectively intersect the top bar 29T″ at the point that upstanding side bars 29U″ intersect the top bar 29T″.

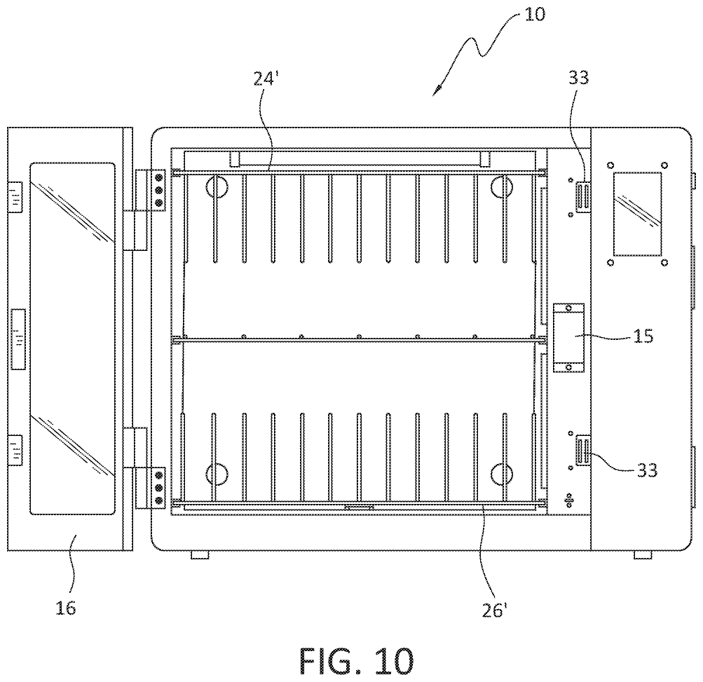

The enclosure further includes a lock 15 that cooperates with the cabinet door 16. The lock 15 operates so that the lock's DEFAULT position is unlocked. This permits the cabinet door 16 to open should there be a power failure. When the power is supplied to the UV lamps 30, 32 the cabinet door 16 is locked. When no power is supplied to the UV lamps 30, 32, the cabinet door 16 may be opened. The lock 15 only goes into the locked position if cabinet door 16 is properly closed. Once properly closed, the cabinet door 16 is locked as soon as the Start button is touched to start supplying power to the UV lamps 30, 32. Once the UV lamps 30, 32 turn off the cabinet door 16 unlocks. The cabinet 10 as best shown in FIG. 10 includes magnets 33 to keep the cabinet door 16 closed when the cabinet 10 is not in use.

In accordance with a disclosed embodiment, the control circuit (not shown) has a countdown or count up timer and either the time remaining until sanitization is displayed on the control panel 18 or the amount of time the UV lamps 30, 32 have been on is displayed. The control panel 18 includes buttons to adjust the amount of time needed to sanitize the contents of the cabinet. The timer can be reset after each operation. It is desired to have the Reset button be in a locked panel so only a technician can access it.

Cabinet 10 has a locking access door 41 on the side where the control panel 18 is located so that a user can access the lock 15 for replacement if needed.

The control panel 18 may include any of the following functions:
1. A default run time of 5 minutes or the required time to clean devices per the UV light manufacturer.
2. Allow administrator to change set time in settings of control panel.
3. Have the total run time of the lamps and a way for the administrator to reset to “0” when lights are changed.
4. Show message “Change Lights in 5 Hours” when Total Run Time gets to 5 hours of time specified by light manufacturer. Have this message count down as the remaining hours reach 4, 3, 2, 1. These numbers will replace the “x” in “Change Lights in ‘x’ Hours.”
5. Show message “Change Lights Now” when Total Run Time gets to time specified by light manufacturer.
6. When the Start button is pushed, the door must lock, and the screen counts down 3 seconds before the lights come on.
7. The ability to communicate with a remote electronic device (for example, an Android® smartphone) via a Wi-Fi network.

While the preferred embodiments have been shown and described, it will be understood that there is no intent to limit the invention by such disclosure, but rather, is intended to coverall modifications and alternate constructions falling within the spirit and scope of the invention.

1. A UV sanitizing cabinet, comprising:
outer walls and a control panel located thereon;
an inner enclosure defined by inner walls and a pivotally mounted door that may be selectively moved between an open orientation and a closed orientation;
an upper rack and a lower rack selectively positioned within the enclosure;
at least one UV lamp secured below the lower rack within the enclosure and at least one UV lamp secured above the upper rack within the enclosure;
a middle shelf selectively positioned within the enclosure; and
tracks secured along the inner walls for supporting the upper and lower racks, as well as the middle shelf, in a manner allowing for selective removal and insertion thereof such that the cabinet can support various electronic devices with minimal contact, including, those with a hinge in an open 180 degree orientation.

2. The cabinet according to claim 1, wherein the inner walls include a left side inner wall, a right side inner wall, an upper inner wall, a lower inner wall, and a back inner wall all made from a highly reflective metal.

3. The cabinet according to claim 2, wherein the at least one UV lamp positioned below the lower rack includes a plurality of staggered lower UV lamps secured along the lower inner wall.

4. The cabinet according to claim 3, wherein the at least one UV lamp position above the upper rack includes a plurality of staggered upper UV lamps secured along the upper inner wall.

5. The cabinet according to claim 2, wherein the at least one UV lamp positioned above the upper rack includes a plurality of upper UV lamps secured along the upper inner wall.

6. The cabinet according to claim 1, wherein the upper rack, the lower rack, and the middle shelf are made from cylindrical wire.

7. The cabinet according to claim 1, wherein the tracks include upper, lower, and middle tracks attached along the left and right inner walls upon which the racks and shelf travel.

8. The cabinet according to claim 1, wherein the upper rack and the lower rack are identical and interchanging.

9. The cabinet according to claim 1, wherein the upper rack and the lower rack include a rectangular wire frame with a plurality of upstanding supports attached along a top of the rectangular wire frame.

10. The cabinet according to claim 1, wherein the middle shelf includes a rectangular wire frame.

11. The cabinet according to claim 1, further including a locking access door on an outer side wall so that a door lock can easily be accessed if needed.

12. The cabinet according to claim 1, wherein the control panel includes a change UV lamp feature which displays a change light message.

13. The cabinet according to claim 9, wherein the plurality of upstanding supports include a single top bar at the peak and upstanding side bars which are bent inwardly prior to respective ends of the single top bar.

14. The cabinet according to claim 13, wherein the plurality of upstanding supports further include riser bars that form a “V” and respectively intersect the top bar at the point that the upstanding side bars intersects the top bar.

15. A UV sanitizing cabinet, comprising:
outer walls and a control panel located thereon;
an inner enclosure defined by inner walls and a pivotally mounted door that may be selectively moved between an open orientation and a closed orientation;
an upper rack and a lower rack selectively positioned within the enclosure, wherein the upper and lower rack are interchangeable;
at least one UV lamp positioned below the lower rack within the enclosure and at least one UV lamp positioned above the upper rack within the enclosure;
a middle shelf selectively positioned within the enclosure; and
tracks secured along the inner walls for supporting the upper and lower racks, as well as the middle shelf, in a manner allowing for selectively removal and insertion thereof such that the cabinet can support various electronic devices with minimal contact, including, those with a hinge in an open 180 degree orientation.

16. The cabinet according to claim 15, wherein the upper rack and the lower rack include a rectangular wire frame with a plurality of upstanding supports attached along a top of the rectangular wire frame.

17. The cabinet according to claim 18, wherein the plurality of upstanding supports include a single top bar at the peak and upstanding side bars which are bent inwardly prior to respective ends of the single top bar.

18. The cabinet according to claim 18, wherein the plurality of upstanding supports further include riser bars that form a “V” and respectively intersect the top bar at the point that the upstanding side bars intersects the top bar

19. The cabinet according to claim 15, wherein the at least one UV lamp positioned below the lower rack includes a plurality of staggered lower UV lamps secured along the lower inner wall.

20. The cabinet according to claim 19, wherein the at least one UV lamp positioned above the upper rack includes a plurality of staggered upper UV lamps secured along the upper inner wall.
